(19) United States
(12) Patent Application Publication
(10) Pub. No.: US 2022/0273016 A1
(43) Pub. Date: Sep. 1, 2022

(54) HUMAN MILK OLIGOSACCHARIDES FOR CONTROL OF DIETARY RESPONSE AND METABOLIC PHENOTYPE

(71) Applicant: The Regents of the University of California, Oakland, CA (US)

(72) Inventors: Daniela BARILE, Davis, CA (US); Sunhye LEE, Davis, CA (US); Helen RAYBOULD, Davis, CA (US)

(21) Appl. No.: 17/627,523

(22) PCT Filed: Jul. 17, 2020
(86) PCT No.: PCT/US2020/042630

§ 371 Date: Jan. 14, 2022

Related U.S. Application Data
(60) Provisional application No. 62/875,912, filed on Jul. 18, 2019.

(51) Int. Cl. A23L 33/00

(52) U.S. Cl. CPC A23L 33/40

(57) ABSTRACT

Provided herein are method for modulating dietary response in a subject. The methods include the administration of human milk oligosaccharides such as 2′-fucosyllactose to the subject. Dietary responses such as decreased adiposity, preservation of lean muscle mass, improved cognitive function, and reduced levels of inflammatory factors can be obtained in various populations, including those consuming high fat diets.

### CROSS-REFERENCES TO RELATED APPLICATIONS

This application claims priority to U.S. Provisional Application No. 62/875,912, filed Jul. 18, 2019, the disclosure of which is hereby incorporated by reference in its entirety for all purposes.

### BACKGROUND OF THE DISCLOSURE

With their multi-functional, health-promoting activities, human milk oligosaccharides (HMOs) have been gaining vast interest not only in the infant formula industry but also for prebiotic applications in adults. At present, a handful of HMOs are being produced industrially, enabling the use of the synthetically produced HMOs as supplements for infant and non-infant population. However, the molecular mechanisms underlying the benefits of HMOs are not yet well understood.

### SUMMARY OF THE DISCLOSURE

In one aspect, the disclosure features a method for modulating dietary response in a subject, in which the method comprises administering an effective amount of a milk oligosaccharide (e.g., human milk oligosaccharide) to the subject. In some embodiments, the modulating dietary response in the subject (e.g., mammals (e.g., human)) comprises: modulating weight gain in the subject, decreasing adiposity in the subject, preserving lean muscle mass in the subject, improving cognitive function in the subject, reducing the level of serum endotoxins in the subject, improving appetite regulation in the subject, decreasing gut permeability, improving gut barrier function, or a combination thereof.

In some embodiments of this aspect, the subject starts with a body mass index (BMI) of 25 or greater prior to administration of the milk oligosaccharide (e.g., human milk oligosaccharide) and reduces the BMI to between 18 and 24.9 (e.g., between 18 and 24, between 18 and 23, between 18 and 22, between 18 and 21, between 18 and 20, between 18 and 19, between 19 and 24, between 20 and 24, between 21 and 24, between 22 and 24, between 23 and 24, between 24 and 24, between 25 and 24, between 26 and 24, between 18 and 24, or between 28 and 24) after administration of the milk oligosaccharide (e.g., human milk oligosaccharide).

In other embodiments, the subject is female and has a body fat percentage of greater than 35% prior to administration of the milk oligosaccharide (e.g., human milk oligosaccharide), and reduces the body fat percentage to 35% or less (e.g., between 25% and 35%, between 25% and 33%, between 25% and 31%, between 25% and 29%, between 25% and 27%, between 27% and 35%, between 29% and 35%, between 31% and 35%, or between 33% and 35%) after administration of the milk oligosaccharide (e.g., human milk oligosaccharide). In other embodiments, the subject is male and has a body fat percentage of greater than 25% prior to administration of the milk oligosaccharide (e.g., human milk oligosaccharide), and reduces the body fat percentage to 25% or less (e.g., between 15% and 25%, between 15% and 23%, between 15% and 21%, between 15% and 19%, between 15% and 17%, between 17% and 25%, between 19% and 25%, between 21% and 25%, or between 23% and 25%) after administration of the milk oligosaccharide (e.g., human milk oligosaccharide).

In some embodiments, the subject scores lower than 24 points on a Mini-Mental State Examination (MMSE) prior to administration of the milk oligosaccharide (e.g., human milk oligosaccharide) and scores 24 points or higher (e.g., between 24 and 30, between 24 and 29, between 24 and 28, between 24 and 27, between 24 and 26, between 24 and 25, between 25 and 30, between 26 and 30, between 27 and 30, between 28 and 30, or between 29 and 30) on the MMSE after administration of the milk oligosaccharide (e.g., human milk oligosaccharide).

In some embodiments of this aspect, the milk oligosaccharide (e.g., human milk oligosaccharide) is selected from the group consisting of 2′-fucosyllactose, 6′-sialyllactose, lacto-N-tetraose, lacto-N-fucopentaose, and combinations thereof. In particular embodiments, the milk oligosaccharide (e.g., human milk oligosaccharide) is 2′-fucosyllactose.

In some embodiments, the subject is a non-infant human. In some embodiments, the subject has a metabolic disorder. For example, the subject can be diabetic, pre-diabetic, or prone to the development of diabetes. In certain embodiments, the subject has gestational diabetes or is prone to the development of gestational diabetes.

In some embodiments, the subject has leaky gut syndrome.

In some embodiments, the subject is elderly, e.g., at least 65 years of age.

In some embodiments, the subject suffers from muscle atrophy, such as muscle atrophy due to age, stunting, or medical conditions.

In some embodiments, the subject exhibits dementia or memory loss prior to administration of the milk oligosaccharide (e.g., human milk oligosaccharide).

In certain embodiments, the subject has sarcopenia. In some embodiments, the subject has excessive muscle atrophy, for example, muscle atrophy that is greater than 5% (e.g., greater than 7%, 9%, 11%, 13%, 15%, 17%, 19%, or 20%) per decade.

In some embodiments, the subject has an elevated level of IL-6 that is greater than 15 pg/mL (e.g., at least 17 pg/mL, at least 19 pg/mL, at least 20 pg/mL, at least 25 pg/mL, at least 30 pg/mL, at least 35 pg/mL, at least 40 pg/mL, at least 45 pg/mL, or at least 50 pg/mL).

In some embodiments, the subject is overweight, e.g., the subject has a body mass index (BMI) between 25 and 30 (e.g., BMI between 25 and 29, BMI between 25 and 28, BMI between 25 and 27, BMI between 25 and 26, BMI between 26 and 29, BMI between 27 and 29, or BMI between 28 and 29). In some embodiments, the subject is obese, e.g., the subject has a body mass index (BMI) of 30.0 or higher (e.g., BMI between 30 and 100, BMI between 30 and 90, BMI between 30 and 80, BMI between 30 and 70, BMI between 30 and 60, BMI between 30 and 50, BMI between 30 and 40, BMI between 40 and 100, BMI between 50 and 100, BMI between 60 and 100, BMI between 70 and 100, BMI between 80 and 100, or BMI between 90 and 100).

In certain embodiments, the subject is a performance athlete. In particular embodiments, the subject has underperformance syndrome (UPS) and/or overtraining syndrome (OTS).

In some embodiments, the subject has autism spectrum disorder.

In certain embodiments, the milk oligosaccharide (e.g., human milk oligosaccharide) is administered to the subject in conjunction with a high-fat diet.

### BRIEF DESCRIPTION OF THE DRAWINGS

FIG. 1A shows body weight in mice fed a low-fat diet (LF), high-fat diet (HF), or high-fat diet+10% 2′-fucosyllactose (HF+10% 2′-FL). 2′-FL was provided in the drinking water for 6 wk. Values are means±SEMs (standard error of the mean), n=6 mice/group. Repeated measures analysis of variance (ANOVA) followed by Tukey's post hoc was performed used to test for differences among groups. Labeled means at a time without a common letter differ, P<0.05.

FIG. 1B shows cumulative energy intake in mice fed a low-fat diet (LF), high-fat diet (HF), or high-fat diet+10% 2′-fucosyllactose (HF+10% 2′-FL). 2′-FL was provided in the drinking water for 6 wk. Values are means±SEMs, n=6 mice/group. One-way ANOVA followed by Tukey's post hoc was performed used to test for differences among groups. Labeled means at a time without a common letter differ, P<0.05. 2′-FL, 2′-fucosyllactose.

FIGS. 2A and 2B show body composition (fat body mass and lean body mass comparison) of mice fed an LF or HF with or without 10% 2′-FL supplementation diet (2′-FL) for 8 weeks. Values are means±SEMs, n=8/group. Repeated measures ANOVA followed by Tukey's post hoc was performed used to test for differences among groups. Asterisk (*) denotes significant differences among groups at *P<0.05, **P<0.01, ***P<0.001, ****P<0.0001. NS, not significant. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 2C shows adipocyte size in visceral adipose tissue of mice fed an LF or HF with or without 10% 2′-FL supplementation diet for 8 weeks. Values are means±SEMs; n=8/group. Two-way ANOVA followed by Tukey's (when the interaction was significant) or Sidak's (for the main effects) post-hoc test was used to test for differences among groups. Asterisk (*) denotes significant differences among groups at *P<0.05, **P<0.01. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 2D shows gene expression of peroxisome proliferator-activated receptor gamma (PPARγ) in the liver of mice fed an LF or HF with or without 10% 2′-FL supplementation diet (2′-FL) for 8 weeks. Values are means±SEMs; n=8/group. Two-way ANOVA followed by Tukey's (when the interaction was significant) or Sidak's (for the main effects) post-hoc test was used to test for differences among groups. Asterisk (*) denotes significant differences among groups at **P<0.01, ***P<0.001. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosylactose.

FIG. 2E shows gene expression of sterol regulatory element binding protein-1c (SREBP-1c) in the liver of mice fed an LF or HF with or without 10% 2′-FL supplementation diet for 8 weeks. Values are means±SEMs; n=8/group. Two-way ANOVA followed by Tukey's (when the interaction was significant) or Sidak's (for the main effects) post-hoc test was used to test for differences among groups. Asterisk (*) denotes significant differences among groups at **P<0.01. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 3A shows food intake levels measured during a cholecystokinin (CCK) feeding study in mice receiving LF, HF, or HF+10% 2′-FL diets for 6 weeks. 2′-FL, 2′-fucosyllactose. Values are means±SEMs, n=6/group. Paired Student's t-test was used to determine statistical significance within groups. Asterisk (*) denotes significant differences among groups at P<0.05. HF, high fat; HF_10% 2′-FL, high fat with 10% 2′-FL (w/v) in drinking water; LF, low fat; 2′-FL, 2′-fucosyllactose.

FIG. 3B shows number of c-Fos immunopositive cells in area postrema (AP) in the hindbrain of mice fed an LF or HF with or without 10% 2′-FL supplementation diet for 8 weeks. Two-way ANOVA followed by Tukey's (when the interaction was significant) or Sidak's (for the main effects) post-hoc test was used to test for differences among groups. Values are means±SEMs; n=8/group. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 3C shows number of c-Fos immunopositive cells in the nucleus of the solitary tract (NTS) in the hindbrain of mice fed an LF or HF with or without 10% 2′-FL supplementation diet for 8 weeks. Values are means±SEMs; n=8/group. Two-way ANOVA followed by Tukey's (when the interaction was significant) or Sidak's (for the main effects) post-hoc test was used to test for differences among groups. Asterisk (*) denotes significant differences among groups at P<0.05. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 4A shows FITC-dextran 4000 (FD4) flux (paracellular pathway) measured in the cecum of mice fed an LF or HF with or without 10% 2′-FL supplementation diet for 8 weeks. Values are means±SEMs; n=8/group. Two-way ANOVA followed by Tukey's (when the interaction was significant) or Sidak's (for the main effects) post-hoc test was used to test for differences among groups. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 4B shows horseradish peroxidase (HRP) flux measured in the cecum of mice fed an LF or HF with or without 10% 2′-FL supplementation diet for 8 weeks. Values are means SEMs; n=8/group. Two-way ANOVA followed by Tukey's (when the interaction was significant) or Sidak's (for the main effects) post-hoc test was used to test for differences among groups. Asterisk (*) denotes significant differences among groups at *P<0.05. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 4C shows gene expression of inerleukin (IL)-22 in the ileum of mice fed an LF or HF with or without 10% 2′-FL supplementation diet for 8 weeks. Values are means±SEMs; n=8/group. Two-way ANOVA followed by Tukey's (when the interaction was significant) or Sidak's (for the main effects) post-hoc test was used to test for differences among groups. Asterisk (*) denotes significant differences among groups at **P<0.01. HF, high fat; HF/CON, HF without 2′-FL; HF/2′-FL, HF with 2′-FL (w/w) in diet; LF, low fat; LF/CON, LF without 2′-FL; LF/2′-FL, LF with 2′-FL (w/w) in diet; 2′-FL, 2′-fucosyllactose.

FIG. 4D shows circulating LPB (lipopolysaccharide-binding protein) levels in mice receiving LF, HF, or HF+10% 2′-FL diets for 6 weeks. Values are means±SEMs, n=6/group. One-way ANOVA followed by Tukey's post hoc was performed used to test for differences among groups. Labeled means without a common letter are significantly different, P<0.05. HF, high fat; HF_10% 2′-FL, high fat with 10% 2′-FL (w/v) in drinking water; LF, low fat; 2′-FL, 2′-fucosyllactose.

FIG. 4E shows gene expression levels of monocyte chemoattractant protein-1 (MCP-1) in adipose tissue in mice receiving LF, HF, or HF+10% 2′-FL diets for 6 weeks. Values are means±SEMs, n=6/group. One-way ANOVA followed by Tukey's post hoc was performed used to test for differences among groups. Labeled means without a common letter are significantly different, P<0.05. HF, high fat; HF_10% 2′-FL, high fat with 10% 2′-FL (w/v) in drinking water; LF, low fat; 2′-FL, 2′-fucosyllactose.

### DETAILED DESCRIPTION OF THE DISCLOSURE

The present disclosure is based, in part, on the discovery that 2′-fucosyllactose (“2′-FL,” the most abundant human breastmilk oligosaccharide) provides beneficial effects in modulating metabolic profiles of metabolically challenged subjects. It has now been discovered, for example, that the supplementation of 2′-FL decreased body weight gain along with energy intake in mice consuming a high-fat diet. Based on the studies described herein, it is believed that 2′-FL treatment restores sensitivity to cholecystokinin (CCK), an anorexigenic gut hormone, which can improve gut-brain signaling and appetite regulation. Further, it was also observed that 2′-FL reduced adiposity while preserving lean mass, mainly by suppressing lipogenesis and adipogenesis transcription factors such as PPARγ and SREBP-1c in the liver. Additionally, it has been shown that there is a connection between inflammation and autism. Studies on autistic children consistently and significantly showed higher concentrations of inflammatory markers, such as interleukin (IL)-1β, IL-6, IL-8, interferon gamma (IFNγ) and monocyte chemotactic protein-1 (MCP-1). As described in more detail below, it has now been discovered that 2′-FL supplementation results in a significant decrease of MCP-1 gene expression in the adipose tissue. In addition, severely autistic adults have significantly higher levels of lipopolysaccharides (also referred to as bacterial endotoxins) circulating in the serum than healthy controls. 2′-FL and other human milk oligosaccharides (HMOs) can therefore be used for alleviating and/or preventing metabolic abnormalities and related conditions such as inflammation, and especially in individuals with metabolic disorders such as obesity and diabetes.

### I. METHODS FOR MODULATING DIETARY RESPONSE

Provided herein are methods for modulating dietary response in a subject. The methods include administering an effective amount of an HMO to the subject. Modulating dietary response in the subject can include: (i) modulating weight gain in the subject; (ii) decreasing adiposity in the subject; (iii) preserving lean muscle mass in the subject; (iv) improving cognitive function in the subject; (v) reducing the level of serum endotoxins in the subject; (vi) improving appetite regulation in the subject; (vii) decreasing gut permeability, (viii) improving gut barrier function; or (ix) a combination of such effects.

HMOs are present in the milk of breastfeeding mothers in high abundance and are unique to mammals and specifically H. sapiens. HMOs are complex glycans; the typical HMO which typically contain lactose at the reducing end of the oligosaccharide and one or more additional L-fucose (Fuc), D-glucose (Glc), D-galactose (Gal), N-acetylglucosamine (GlcNAc), and/or N-acetyl neuraminic acid (NeuAc or Neu5Ac; also referred to as sialic acid or Sia) subunits. HMOs range in size from trisaccharides (e.g., 2′-fucosyllactose and 6′-sialyllactose) to polysaccharides. See, e.g., Kunz, C. et al., Annual. Rev. Nutri. (2000) 20:699-722.

Examples of HMOs include, but are not limited to, 2′-fucosyllactose (2′-FL); 3′-fucosyllactose (3′-FL); 3′-sialyllactose (3′-SL); 6′-sialyllactose (6′-SL); lacto-N-tetraose (LNT); lacto-N-neotetraose (LNnT); lacto-N-fucopentaose I; lacto-N-fucopentaose II; lacto-N-fucopentaose III; lacto-N-fucopentaose V; lacto-N-hexaose; para-lacto-N-hexaose; lacto-N-neohexaose; para-lacto-N-neohexaose; monofucosyllacto-N-hexaose II; isomeric fucosylated lacto-N-hexaose (1); monofucosyllacto-N-hexaose; isomeric fucosylated lacto-N-hexaose (3); isomeric fucosylated lacto-N-hexaose (2); difucosyl-para-lacto-N-neohexaose; difucosyl-para-lacto-N-hexaose; difucosyllacto-N-hexaose; lacto-N-neoocataose; para-lacto-N-octaose; iso-lacto-N-octaose; lacto-N-octaose; monofucosyllacto-N-neoocataose; monofucosyllacto-N-octaose; difucosyllacto-N-octaose I; difucosyllacto-N-octaose II; difucosyllacto-N-neoocataose II; difucosyllacto-N-neoocataose I; lacto-N-decaose; trifucosyllacto-N-neooctaose; trifucosyllacto-N-octaose; and trifucosyl-iso-lacto-N-octaose. Any one or combination of such HMOs may be administered to the subject in the methods of the present disclosure.

In some embodiments, the HMO administered to the subject is selected from the group consisting of 2′-fucosyllactose (2′-FL), 6′-sialyllactose (6′-SL), lacto-N-tetraose (LNT), lacto-N-fucopentaose, and combinations thereof. In some embodiments, the HMO is 2′-fucosyllactose.

Subjects contemplated for treatment with the methods described herein include, but are not limited to, humans, companion animals, and livestock. In some embodiments, the subject is a non-infant human. The non-infant human may be, for example, at least 1 year of age, at least 5 years of age, at least 10 years of age, at least 20 years of age, at least 30 years of age, at least 40 years of age, at least 50 years of age, or older. In some embodiments, the subject is elderly. Elderly subjects may be, for example, at least 65 years of age, or at least 75 years of age, or at least 85 years of age. In some embodiments, the subject suffers from muscle atrophy, such as muscle atrophy due to age, stunting, or medical conditions.

In some embodiments, the subject has a metabolic disorder. The subject may be, for example, diabetic or pre-diabetic. The term “diabetic” refers to subject exhibiting hyperglycemia (high blood glucose) due to insulin resistance in the case of type 2 diabetes, or due to reduced insulin production in the case of type 1 diabetes and type 2 diabetes. The term “pre-diabetic” refers to subject showing higher blood glucose level than normal ranges but not high enough to be diagnosed as type 2 diabetes. A pre-diabetic condition increases the risk of developing type 2 diabetes and other metabolic disorders, such as heart disease and stroke.

In some embodiments, the subject has gestational diabetes. Subjects prone to developing diabetes (e.g., persons with elevated blood glucose levels and/or elevated hemoglobin A1c levels) may also be treated with the methods described herein.

In some embodiments, the subject has leaky gut syndrome. Leaky gut syndrome refers to a condition where the tight junctions of intestinal walls become loose, which makes the gut become more permeable to allow bacteria and other toxins to pass from the gut into the bloodstream. As shown in Example 4, supplementation with 2′-FL was demonstrated to decrease paracellular permeability.

In some embodiments, the subject is overweight, e.g., the subject has a body mass index (BMI) between 25 and 30. In some embodiments, the subject is obese. Typically, an obese adult will exhibit a body mass index (BMI) of 30.0 or higher. Typically, an obese child will exhibit a BMI at or above the 85^{th} percentile for children of the same age and sex. The BMI of an individual subject can be determined by dividing the subject's weight (in kilograms) by the square of the subject's height (in meters). Obesity in a female subject, in particular, can predispose the subject to the development of gestational diabetes. Maternal obesity is also strongly associated with offspring obesity; gestational hyperglycemia and subsequent fetal hyperinsulinemia can increase adiposity, impaired glucose tolerance, hyperinsulinemia, and insulin resistance in the offspring. Supplementation with HMOs such as 2′-FL can reduce adiposity and reduce body weight, as demonstrated experimentally below. Therefore administration of HMOs to women during pre-pregnancy (e.g., during preconception counseling) or pregnancy can provide for the mitigation or prevention of maternal obesity, the treatment or avoidance of gestational diabetes, and the protection of the health of the offspring during pregnancy, infancy, and childhood. In these and other embodiments, the response of the subject to her dietary intake can include controlled weight gain upon treatment according to the methods of the present disclosure.

In some embodiments of the methods disclosed herein, the subject starts with a body mass index (BMI) of 30 or greater prior to administration of the human milk oligosaccharide and reduces the BMI to between 18 and 29.9 (e.g., between 18 and 29, between 18 and 28, between 18 and 27, between 18 and 26, between 18 and 25, between 18 and 24, between 18 and 23, between 18 and 22, between 18 and 21, between 18 and 20, between 18 and 19, between 19 and 29, between 20 and 29, between 21 and 29, between 22 and 29, between 23 and 29, between 24 and 29, between 25 and 29, between 26 and 29, between 18 and 29, or between 28 and 29) after administration of the human milk oligosaccharide.

In other embodiments, the subject is female and has a body fat percentage of greater than 35% prior to administration of the human milk oligosaccharide, and reduces the body fat percentage to 35% or less (e.g., between 25% and 35%, between 25% and 33%, between 25% and 31%, between 25% and 29%, between 25% and 27%, between 27% and 35%, between 29% and 35%, between 31% and 35%, or between 33% and 35%) after administration of the human milk oligosaccharide. In other embodiments, the subject is male and has a body fat percentage of greater than 25% prior to administration of the human milk oligosaccharide, and reduces the body fat percentage to 25% or less (e.g., between 15% and 25%, between 15% and 23%, between 15% and 21%, between 15% and 19%, between 15% and 17%, between 17% and 25%, between 19% and 25%, between 21% and 25%, or between 23% and 25%) after administration of the human milk oligosaccharide. Methods and techniques to measure body fat percentage are available, for example, as described in Swaison et al., PLoS One 12(5):e0177175, 2017; and O'Neill et al., Int J Sports Med 37(5):359-63, 2016.

In some embodiments, the subject scores lower than 24 points on a Mini-Mental State Examination (MMSE) prior to administration of the human milk oligosaccharide and scores 24 points or higher (e.g., between 24 and 30, between 24 and 29, between 24 and 28, between 24 and 27, between 24 and 26, between 24 and 25, between 25 and 30, between 26 and 30, between 27 and 30, between 28 and 30, or between 29 and 30) on the MMSE after administration of the human milk oligosaccharide. The Mini-Mental State Examination (MMSE) is a 30-point questionnaire that is used extensively in clinical and research settings to measure cognitive impairment and has also been used in the diagnosis of autism spectrum disorder, (Raja & Azzoni, Psychiatria Danubina, 22(4):514-521, 2010; Pangman et al., Applied Nursing Research. 13(4):209-213, 2000). It is commonly used in medicine and allied health to screen for dementia. It is also used to estimate the severity and progression of cognitive impairment and to follow the course of cognitive changes in an individual over time; thus making it an effective way to document an individual's response to treatment. In some embodiments, a score of 24 or more (out of 30) indicates a normal cognition. Below this, scores can indicate severe (<9 points), moderate (10-18 points), or mild (19-23 points) cognitive impairment.

Administration of HMOs such as 2′-FL can decrease the production of inflammatory factors that are associated with obesity and other conditions. Such conditions include, but are not limited to, autism spectrum disorder (ASD) which may manifest as conditions such as autism, Asperger's syndrome, or childhood disintegrative disorder. Autistic children have significantly higher concentrations of interleukin (IL)-1β, IL-6, IL-8, interferon gamma (IFNγ) and monocyte chemotactic protein-1 (MCP-1). As described in more detail below, it has now been discovered that 2′-FL supplementation results in a significant decrease of MCP-1 gene expression in the adipose tissue. In addition, severely autistic adults have significantly higher levels of lipopolysaccharides (also referred to as bacterial endotoxins) circulating in the serum than healthy controls. During development of the methods described herein, it was discovered that 2′-FL supplementation can reduce the circulating level of lipopolysaccharide-binding protein (LBP) which can, in turn, reduce serum endotoxin levels. By reducing systemic inflammation through the administration of HMOs such as 2′-FL, neuroinflammation can be reduced and cognitive improvements can be achieved in subjects having ASD or other individuals with obesity-linked cognitive defects. In these and other embodiments, the response of subjects to their dietary intake can include reduced weight gain, decreased adiposity, improved cognitive function, or a combination thereof upon treatment according to the methods of the present disclosure.

Elderly subjects may exhibit conditions such as reduced skeletal muscle mass (normal muscle atrophy or at severe cases, sarcopenia), dementia, and/or memory loss. Muscle weakness in older adults, and more particularly, sarcopenia, the age-associated loss of skeletal muscle mass in elderly persons, is correlated with and increased levels of circulating inflammatory factors including cytokines such as interleukin (IL)-IL-6 (e.g., an elevated level of IL-6 that is greater than 15 pg/mL). In some embodiments, the subject is elderly and exhibits sarcopenia. In some embodiments, the subject has excessive muscle atrophy, for example, muscle atrophy that is greater than 5% (e.g., greater than 7%, 9%, 11%, 13%, 15%, 17%, 19%, or 20%) per decade. In some embodiments, the subject is elderly and exhibits dementia or memory loss prior to administration of the HMO. In certain instances, obesity can contribute to the exacerbation of age-related conditions such as sarcopenia and dementia. Reducing inflammation by administering HMOs according to the methods of the present disclosure can counteract the effect of obesity in elderly subjects. In these and other embodiments, the response of subjects to their dietary intake can include reduced weight gain, decreased adiposity, improved cognitive function, preservation of lean muscle mass, or a combination thereof upon treatment according to the methods of the present disclosure.

Subjects treated with the methods provided herein need not be prone to obesity, cognitive defects, or other diseases. In some embodiments, for example, the subject may be athlete, e.g., a high-performance athlete or an endurance athlete such as a gymnast or long-distance runner. Modulation of dietary response in such athletes can reduce inflammation associated with cytokines released during and after prolonged exercise which, in turn, can reduce the likelihood of underperformance syndrome (UPS) and/or overtraining syndrome (OTS). UPS and OTS refer to a persistent decrement in athletic performance capacity in a subject, despite 2 weeks of rest. Cytokine IL-6 levels, for example, have been observed to increase by more than 120-fold following a marathon race. In some embodiments, the subject is an athlete who has an elevated level of IL-6 that is greater than 15 pg/mL. Endotoxemia, i.e., elevated circulating levels of endotoxin, has also been correlated with increased TNFα, IL-1β, and IL-6 levels. Reducing inflammation and reducing endotoxemia by administering HMOs according to the methods of the present disclosure therefore can counteract the effect of underperformance syndrome or overtraining syndrome.

In some embodiments, the HMO is administered to the subject in conjunction with a high-fat diet. As used herein the term “high-fat” refers to a diet wherein at least 30% (e.g., at least 35%, at least 40%, at least 45%, or at least 50%) of the calories consumed by an individual are obtained from fats. High fat diets increase intestinal permeability and cause dysbiosis, promoting both local and systemic inflammation. Problems resulting from such inflammation can be improved or resolved using the methods of the present disclosure.

In some embodiments, the HMO can be administered to the subject in an amount less than 20 gram per day (e.g., between 0.1 g/day and 20 g/day, between 0.1 g/day and 18 g/day, between 0.1 g/day and 16 g/day, between 0.1 g/day and 14 g/day, between 0.1 g/day and 12 g/day, between 0.1 g/day and 10 g/day, between 0.1 g/day and 8 g/day, between 0.1 g/day and 6 g/day, between 0.1 g/day and 4 g/day, between 0.1 g/day and 2 g/day, between 0.1 g/day and 1 g/day, between 1 g/day and 20 g/day, between 2 g/day and 20 g/day, between 4 g/day and 20 g/day, between 6 g/day and 20 g/day, between 8 g/day and 20 g/day, between 10 g/day and 20 g/day, between 12 g/day and 20 g/day, between 14 g/day and 20 g/day, between 16 g/day and 20 g/day, or between 18 g/day and 20 g/day). The HMO may be administered once per day or in multiple doses to obtain the total daily dose. The terms “about” and “around,” as used herein to modify a numerical value, indicate a close range surrounding that explicit value. If “X” were the value, “about X” or “around X” would indicate a value from 0.9X to 1.1X, and in certain instances, a value from 0.95X to 1.05X or from 0.98X to 1.02X, or from 0.99X to 1.01X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.99X.”

HMOs may be formulated into pills, tablets, encapsulated in capsules, such as gelatin capsules, liquid shake, bars, functional food, gummies, and gels etc. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenges and like forms can comprise flavorings, e.g., sucrose, and inert base materials, such as gelatin and glycerin or sucrose and acacia emulsions. HMO compositions may also contain conventional food supplement fillers and extenders such as, for example, rice flour. In some embodiments, the HMO is administered in a composition containing one or more non-human proteins, non-human lipids, non-human carbohydrates, or other non-human components. For example, in some embodiments, the HMO is administered in a composition containing bovine (or other non-human) milk protein, a soy protein, betalactoglobulin, whey, soybean oil, or starch. Alternatively, HMOs may be added directly to food items including, but not limited to, dairy-based products such as cheese, cottage cheese, yogurt, and ice cream; beverages such as water, coffee, tea, infant formula, milk, fermented milk, fruit juice, fruit-based drinks, and sports drinks; and plant-based meat substitutes such as those described in WO 2015/153666 and US 2015/0289541. In addition to foods targeted for human consumption, animal feeds may also be supplemented with HMOS for use in the methods.

The HMOs used in the methods of the disclosure can be derived using any of a number of sources and methods known to those of skill in the art. For example, HMOs can be purified from human milk using methods known in the art. One such method for extraction of oligosaccharides from pooled mother's milk entails the centrifugation of milk at 5,000×g for 30 minutes at 4° C. and fat removal. Ethanol is then added to precipitate proteins. After centrifugation to sediment precipitated protein, the resulting solvent is collected and dried by rotary evaporation. The resulting material is adjusted to the appropriate pH of 6.8 with phosphate buffer and β-galactosidase is added. After incubation, the solution is extracted with chloroform-methanol, and the aqueous layer is collected. Monosaccharides and disaccharides are removed by selective adsorption of HMOs using solid phase extraction with graphitized nonporous carbon cartridges. The retained oligosaccharides can be eluted with water-acetonitrile (60:40) with 0.01% trifluoroacetic acid. (See, e.g., Ward et al., Appl. Environ. Microbiol. (2006), 72: 4497-4499; Gnoth et al., J. Biol. Chem. (2001), 276:34363-34370; Redmond and Packer, Carbohydr. Res. (1999), 319:74-79.) Individual HMOs can be further separated using methods known in the art such as HPLC (e.g., high-performance anion-exchange chromatography with pulsed amperometric detection; HPAEC-PAD), and thin layer chromatography. See, e.g., Splechtna et al., J. Agricultural and Food Chemistry (2006), 54: 4999-5006.

Alternatively, enzymatic methods can be used to synthesize the HMOs of the present disclosure. For example, galacto-oligosaccharides have been synthesized from lactose using the β-galactosidase from L. reuteri (see, Splechtna et al., J. Agricultural and Food Chemistry (2006), 54: 4999-5006). The reaction employed is known as transgalactosylation, whereby the enzyme β-galactosidase hydrolyzes lactose, and, instead of transferring the galactose unit to the hydroxyl group of water, the enzyme transfers galactose to another carbohydrate to result in oligosaccharides with a higher degree of polymerization (Vandamme and Soetaert, FEMS Microbiol. Rev. (1995), 16:163-186). A related method utilizes the β-galactosidase of Bifidobacterium bifidum NCIMB 41171 to synthesize galacto-oligosaccharides (see, Tzortzis et al., Appl. Micro. and Biotech. (2005), 68:412-416). HMOs may also be expressed in recombinant organisms such as E. coli or S. cerevisiae. See, e.g., Baumgärtner, et al. Microb Cell Fact. (2013), 12: 40; Chin, et al. J Biotechnol. (2015), 210: 107-115; and Lee, et al. Microb Cell Fact. (2012), 11: 48.

Another approach to the synthesis of the carbohydrates of the disclosure that combines elements of the methods outlined above entails the chemical or enzymatic synthesis of or isolation of oligosaccharide backbones containing lacto-N-biose, or lacto-N-tetraose from non-human mammalian milk sources (e.g., cows, sheep, buffalo, goat, etc.) and enzymatically adding lacto-N-biose, fucose, and sialic acid units as necessary to arrive at the HMO structures of the present disclosure. For this purpose, a variety of carbohydrate modifying enzymes, such as those disclosed in WO 2008/033520 can be utilized. Examples of such oligosaccharide modifying enzymes include sialidases, sialyl O-acetylesterases, N-acetylneuraminate lyases, N-acetyl-beta-hexosaminidases, beta-galactosidases, N-acetylmannosamine-6-phosphate 2-epimerases, alpha-L-fucosidases, and fucose dissimilation pathway proteins, among others.

Another approach to the synthesis of the carbohydrates of the disclosure is via fermentation by whole-cell biotransformation (see, e.g., Sprenger et al. J. Biotechnology, 258:79-91, 2017) with recombinant bacterial cells (e.g., E. coli) and further purification from the media/broth (e.g., by selective crystallization—see, e.g., WO2020079114A1)

### II. EXAMPLES

### Example 1. Consumption of 2′-Fucosyllactose with a High-Fat Diet Decreases Weight Gain and Energy Intake

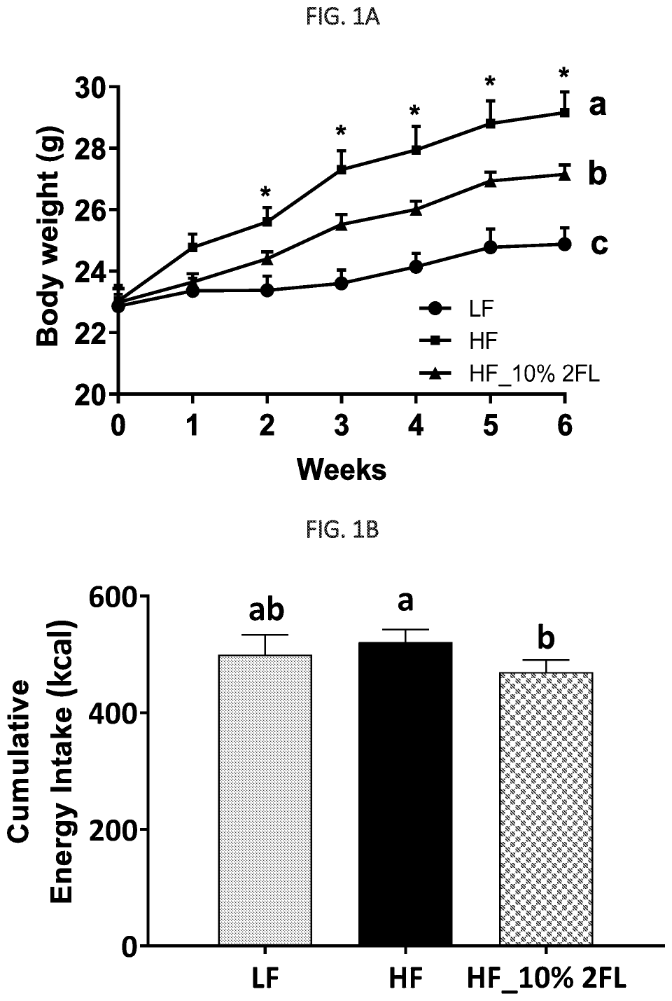

Animals were maintained and handled in accordance with protocols approved by the Institutional Animal Care and Use Committee (University of California, Davis). Male C57/BL6 mice (n=6/group; 6 week old, Harlan, San Diego, Calif.) were split into three weight-matched groups and fed either a low-fat (LF; 10% kcal as fat), high-fat (HF; 45% kcal as fat), or HF diet with 2′-FL (HF_2′-FL) at 10% (w/v) in drinking water for 6 wk. All animals were housed individually at 22° C. with 12 h:12 h light-dark cycles. Body weight and food intake were measured twice a week. Statistical analysis was performed using Prism software (Prism 8.1.2; GraphPad Software). Six-week HF feeding increased energy intake compared to the LF groups but without statistical significance (FIG. 1B). However, this increase was suppressed by 10% 2′-FL supplementation, leading to a significantly difference lower body weight compared with the HF group (FIG. 1A). For FIGS. 1A and 1B, data are presented as means±SEMs. ROUT test was used to exclude outliers. Differences were considered significant if P<0.05.

### Example 2. Consumption of 2′-Fucosyllactose Decreases Adiposity while Preserving Lean Mass

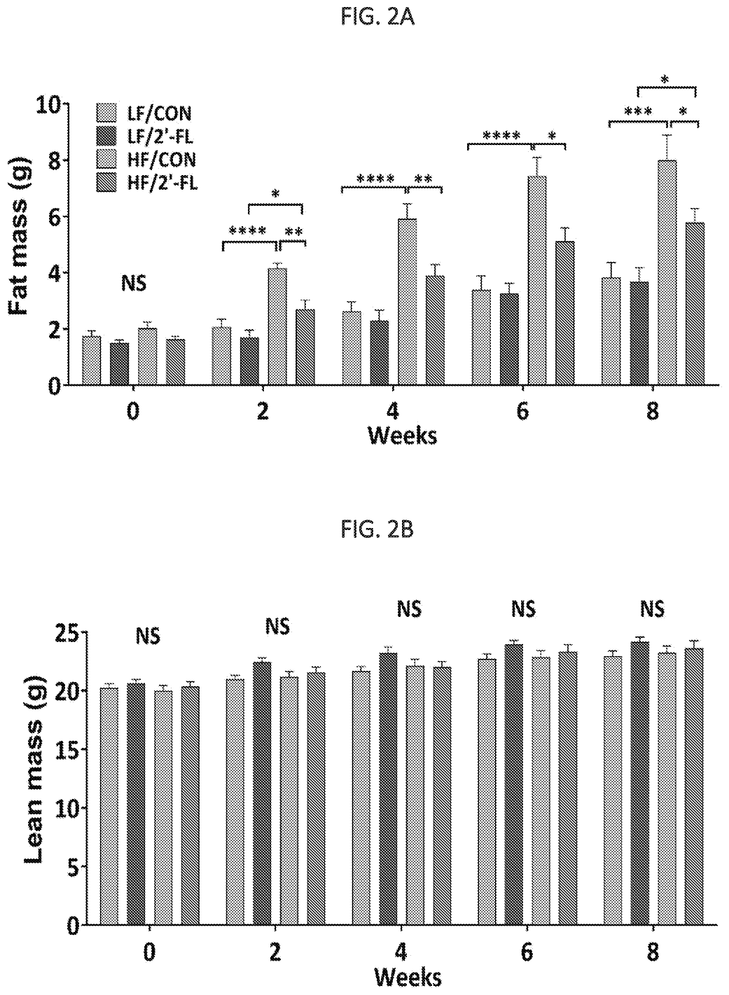

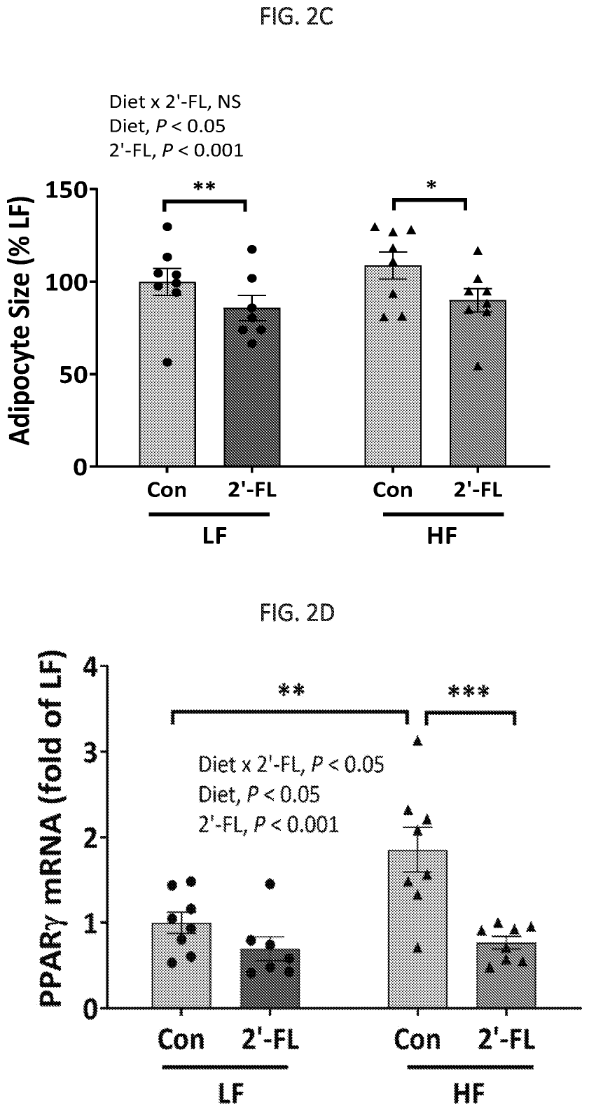

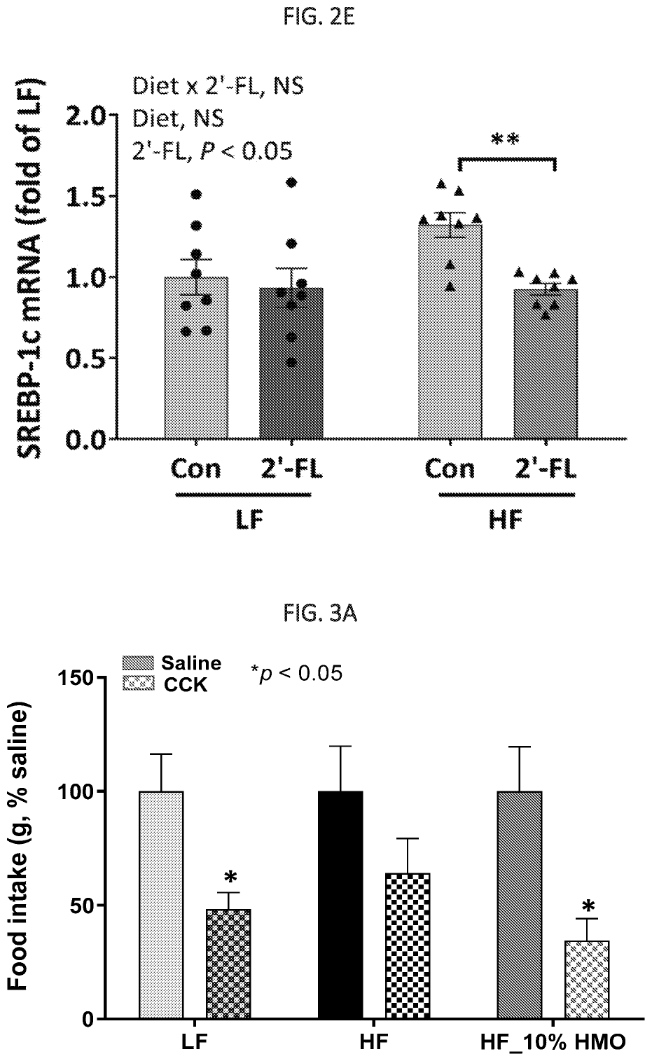

Body composition for fat and lean mass (FIGS. 2A and 2B) was analyzed in live animals using EchoMRI-100 ™ from Echo Medical Systems (Houston, Tex.) every two weeks in mice fed an LF or HF with or without 10% 2′-FL supplementation. To measure the size of adipocytes (FIG. 2C), mesenteric adipose tissue collected at euthanasia was fixed in 10% neutral-buffered formalin (Thermo Fisher Scientific, Waltham, Mass.) overnight and transferred to 70% ethanol for one day. Afterwards, the tissues were processed in a routine manner for paraffin sections (Tissue Tek VIP Tissue Processor; Sakura Finetek USA, Torrance, Calif.). Paraffin-embedded sections (5 μm) were cut and stained with H&E (Sigma-Aldrich) for microscopic examination (Olympus BX60, Waltham, Mass.) at 20× magnification. To quantitate adipocyte size (FIG. 2C), the H&E-stained sections were analyzed using the ImageJ software (National Institutes of Health, Bethesda, Md.). For hepatic gene expression of peroxisome proliferator-activated receptor γ (PPAR γ) and sterol regulatory element binding protein-1c (SREBP-1c) (FIGS. 2D and 2E), total RNA from liver samples was extracted via the TRIzol method (Life Technologies, Grand Island, N.Y.). cDNAs were synthesized from 1 pg of purified RNA samples using iScript cDNA synthesis kit (Bio-Rad, Hercules, Calif.) following the manufacturer's protocol. Real-time PCR was performed with the Quantstudio 6 Flex real-time PCR machine using SyberGreen master mix (Life Technologies) for detection. R-Actin and GAPDH were used as housekeeping genes. Genes of interest were analyzed according to the 2^{-ΔΔT} method and compared with control samples.

The results indicate that 2′-FL supplementation suppresses adipogenesis and downregulates gene expression of PPAR γ and SREBP-1c in the liver. For FIGS. 2A-2E, statistical analysis was performed by using Prism software (Prism 8.4; GraphPad Software, La Jolla, Calif.). Data are presented as means±SEMs. ROUT test was used to exclude outliers. Differences were considered significant if P<0.05.

### Example 3. Consumption of 2′-Fucosyllactose Improves Appetite Regulation Via Gut-Brain Signaling

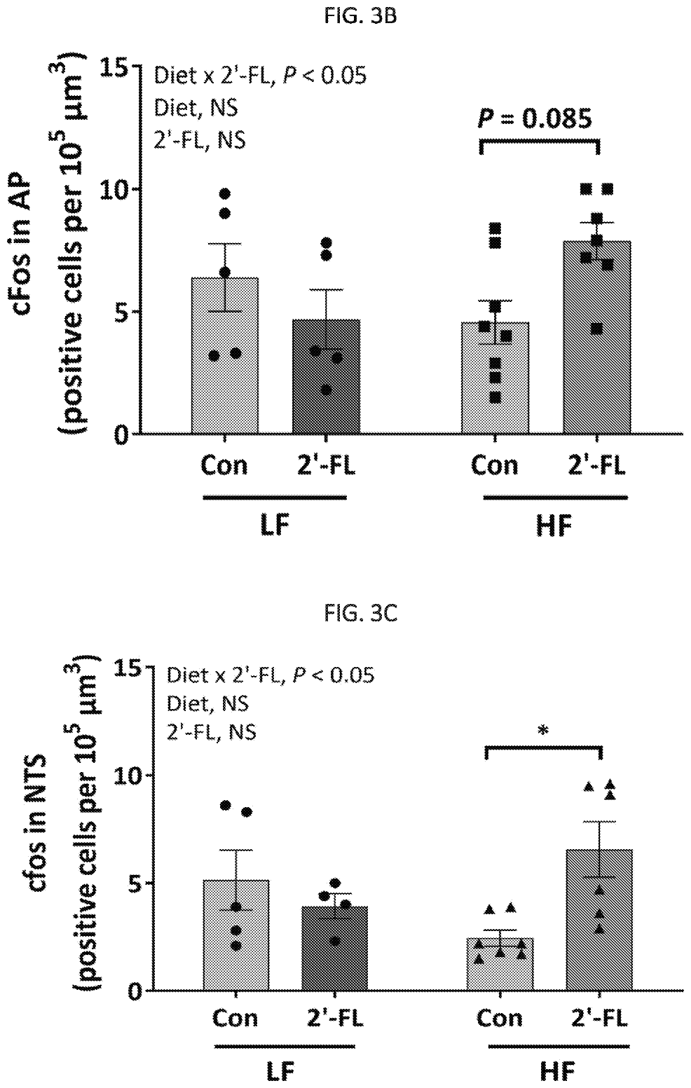

After 4 weeks on respective diets, 2′-FL sensitivity to the satiating effect of the gut peptide CCK was tested (FIG. 3A). Experiments were performed at the onset of the dark phase. Mice were fasted on wire-bottom cages for 6 h during the light phase. At the onset of the dark phase, CCK (octapeptide, sulfated, Bachem, Torrance, Calif., 100 μl at 3 pg/kg; i.p.) or saline (100 μl; i.p.) was administered. Food was placed in the cage and food intake recorded after 20 min. After 6 weeks of respective diets, c-Fos expression in hindbrain was determined. (FIGS. 3B and 3C). Mice were fasted for 4 h, ip injected with CCK (20 pg/kg), and euthanized using deep anesthesia induced with pentobarbital (Fatal Plus, Vortech Pharmaceuticals, Dearborn, Mich., USA; 300 mg/kg; i.p.). The hindbrain was collected and post-fixed in 4% paraformaldehyde (Electron Microscopy Sciences, Hatfield, Pa.). Hindbrains were cryosectioned at 30 μm thickness and stained for c-Fos protein expression in the NTS and AP regions. Sections were permeabilized in PBST (phosphate-buffered saline containing 0.10% Tween 20, Sigma-Aldrich), blocked in 5% normal goat serum in 0.2% Triton X-100 (Sigma-Aldrich) in PBST for 1 hour at room temperature, and incubated overnight in a primary antibody (c-Fos at 1:100; Cell Signaling Technology, Beverly, Mass.) at 4° C. After washes, signals were revealed by incubation with a secondary antibody (1:500; Alexa Fluor 647, Invitrogen, Carlsbad, Calif.) in blocking buffer for 1 hour in the dark at room temperature. For visualization of vagal afferents, sections were incubated with isolectin GS-IB4 Alexa Fluor 594 (IB4, 1:500; Molecular Probes, Eugene, Oreg.). Nuclei were counterstained with 4′,6-diamidino-2-phenylindole (DAPI, 1:5,000; Invitrogen) for 5 min followed by washes. Sections mounted on slides were closed with Prolong antifade mounting medium (Molecular Probes). Images were acquired using a confocal microscope (Leica TCS SP8 STED 3×; Leica, Wetzlar, Germany) and quantified in a blinded manner using Imaris Software (Bitplane, Zurich, Switzerland).

The results indicated that 2′-FL supplementation restores sensitivity to CCK (FIG. 3A) and restores intestinal satiety signals (FIGS. 3B and 3C). For FIGS. 3A-3C, statistical analysis was performed by using Prism software (Prism 8.4; GraphPad Software, La Jolla, Calif.). Data are presented as means±SEMs. ROUT test was used to exclude outliers. Differences were considered significant if P<0.05.

### Example 4. Consumption of 2′-Fucosyllactose Modulates Inflammatory Profiles

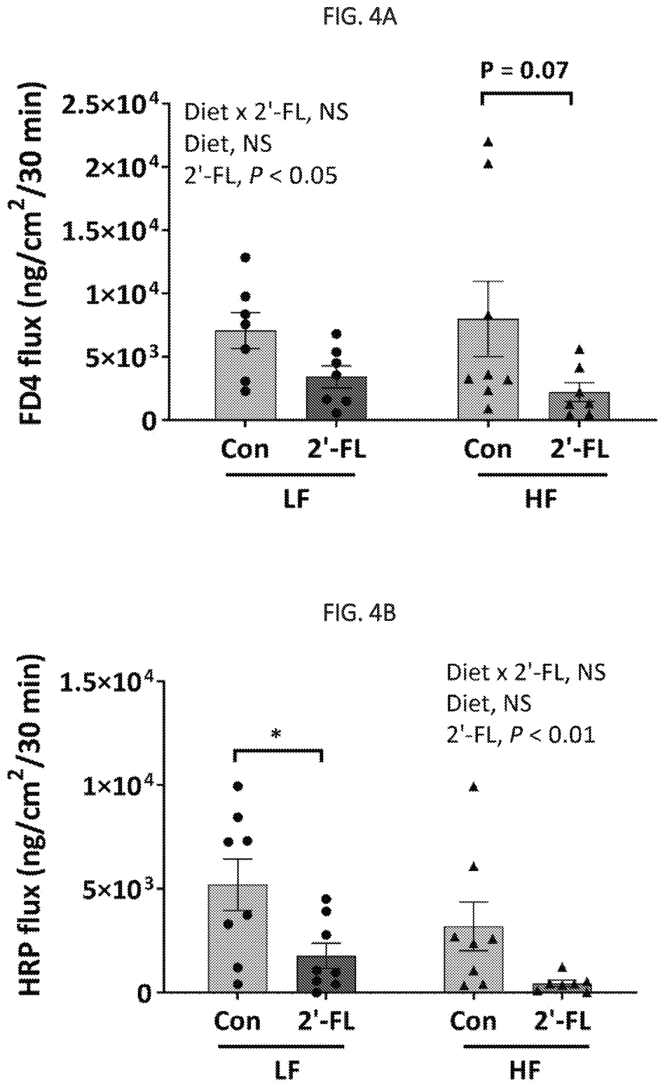

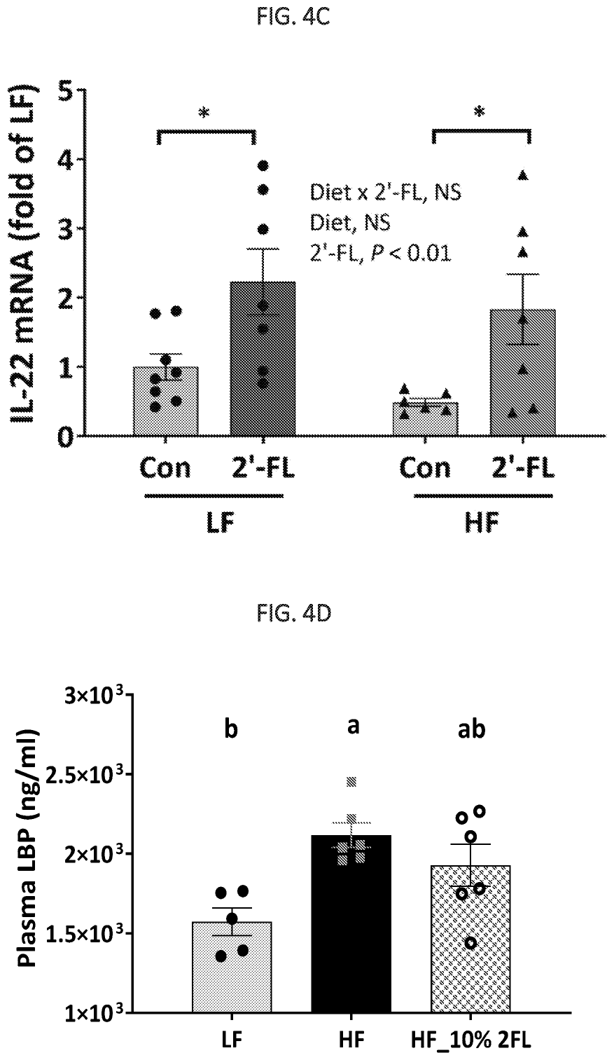

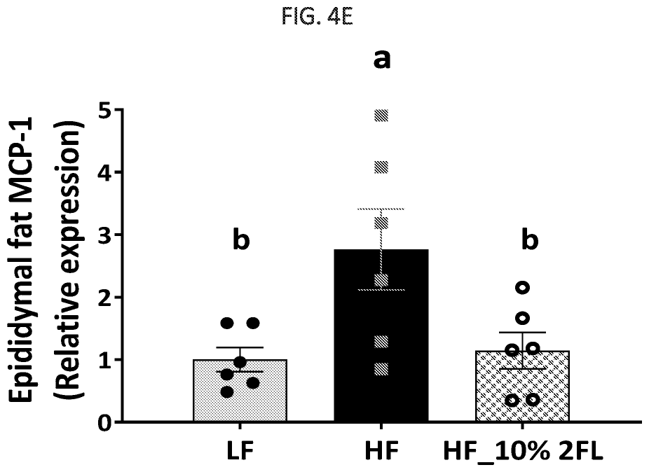

To measure gut permeability (FIGS. 4A and 4B), a section of cecum was opened along the mesenteric border and mounted in Ussing chambers (Physiologic Instruments, San Diego, Calif.), exposing 0.3 cm² of tissue surface area to 2.5 ml of oxygenated Krebs-glucose (10 mM) and Krebs-mannitol (10 mM) at 37° C. on the serosal and luminal sides, respectively. The paracellular pathway and transcellular pathway were measured as the flux of FITC-4000 (FD-4; Sigma-Aldrich) and horseradish peroxidase (HRP Type VI; Sigma Aldrich), respectively. FD-4 (400 pg/ml) and HRP (200 pg/ml) were added to the mucosal chamber, and samples were collected from the serosal chamber every 30 min for 2 h. The concentration of FD-4 was measured via fluorescence at excitation of 485 nm and emission of 538 nm. O-dianisidine substrate was used to detect HRP at absorbance 450 nm. Data are shown as flux at 90 min. Gene expression of IL-22 in the ileum (FIG. 4C) and MCP-1 (FIG. 4E) in adipose tissue were measured as described in Example 2. LBP levels (FIG. 4D) were detected in plasma via enzyme-linked immunosorbent assay according to manufacturer's recommendation (Biometec).

The results indicated that 2′-FL supplementation decreased paracellular permeability, reduced circulating LPB levels, and systemically lowered expression of inflammatory cytokines in adipocytes. For FIGS. 4A-4E, statistical analysis was performed by using Prism software (Prism 8.4; GraphPad Software, La Jolla, Calif.). Data are presented as means SEMs. ROUT test was used to exclude outliers. Differences were considered significant if P<0.05.

Although the foregoing has been described in some detail by way of illustration and example for purposes of clarity and understanding, one of skill in the art will appreciate that certain changes and modifications can be practiced within the scope of the appended claims. All publications, patents, patent applications, and sequence accession numbers cited herein are hereby incorporated by reference in their entirety for all purposes.

1. A method for modulating dietary response in a subject, the method comprising administering an effective amount of a milk oligosaccharide to the subject.

2. The method of claim 1, wherein modulating dietary response in the subject comprises:
modulating weight gain in the subject,
decreasing adiposity in the subject,
preserving lean muscle mass in the subject,
improving cognitive function in the subject,
reducing the level of serum endotoxins in the subject,
improving appetite regulation in the subject,
decreasing gut permeability,
improving gut barrier function, or
a combination thereof.

3. The method of claim 1, wherein the subject starts with a body mass index (BMI) of 25 or greater prior to administration of the milk oligosaccharide and reduces the BMI to between 18 and 29.9 after administration of the milk oligosaccharide.

4. The method of claim 1, wherein the subject is female and has a body fat percentage of greater than 35% prior to administration of the milk oligosaccharide, and reduces the body fat percentage to 35% or less after administration of the milk oligosaccharide.

5. The method of claim 1, wherein the subject is male and has a body fat percentage of greater than 20% prior to administration of the milk oligosaccharide, and reduces the body fat percentage to 20% or less after administration of the milk oligosaccharide.

6. The method of claim 1, wherein the subject scores lower than 24 points on a Mini-Mental State Examination (MMSE) prior to administration of the milk oligosaccharide and scores 24 points or higher on the MMSE after administration of the milk oligosaccharide.

7. The method of claim 1, wherein the milk oligosaccharide is selected from the group consisting of 2′-fucosyllactose, 6′-sialyllactose, lacto-N-tetraose, lacto-N-fucopentaose, and combinations thereof.

8. The method of claim 7, wherein the milk oligosaccharide is 2′-fucosyllactose.

9. The method of claim 1, wherein the subject is a non-infant human.

10. The method of claim 1, wherein the subject has a metabolic disorder.

11. The method of claim 1, wherein the subject is diabetic, pre-diabetic, or prone to the development of diabetes; and/or
wherein the subject has gestational diabetes or is prone to the development of gestational diabetes.

12. The method of claim 11, wherein the subject has gestational diabetes or is prone to the development of gestational diabetes.

13. The method of claim 1, wherein the subject has leaky gut syndrome.

14. The method of claim 1, wherein the subject is elderly; and/or
wherein the subject suffers from muscle atrophy due to age, stunting or medical conditions.

15. The method of claim 13, wherein the subject suffers from muscle atrophy due to age, stunting or medical conditions.

16. The method of claim 1, wherein the subject exhibits dementia or memory loss prior to administration of the milk oligosaccharide; and/or
wherein the subject has excessive muscle atrophy that is greater than 5% per decade; and/or
wherein the subject has an elevated level of IL-6 that is greater than 15 pg/mL.

17. The method of claim 1, wherein the subject has excessive muscle atrophy that is greater than 5% per decade.

18. The method of claim 1, the subject has an elevated level of IL-6 that is greater than 15 pg/mL.

19. The method of claim 1, wherein the subject is obese.

20. The method of claim 19, wherein the subject has a BMI of 30.0 or higher.

21. The method of claim 1, wherein the subject is a performance athlete.

22. The method of claim 21, wherein the subject has underperformance syndrome (UPS) and/or overtraining syndrome (OTS).

23. The method of claim 1, wherein the subject has autism spectrum disorder.

24. The method of claim 1, wherein the milk oligosaccharide is administered to the subject in conjunction with a high-fat diet.

25. The method of claim 1, wherein the milk oligosaccharide is a human milk oligosaccharide.

26. The method of claim 1, wherein the subject is an animal.

27. The method of claim 26, wherein the subject is a companion animal or livestock animal.

28. The method of claim 26, wherein the animal exhibits decreased fat mass and preserved lean muscle mass relative to control.

29. An animal resulting from the method of claim 28.
